SEQUENCE LISTING (10 sequences)

SEQ ID NO: 1 - DNA, 1089 bp, Homo sapiens
atggcagctgctgaggaggagccgaagcccaaaaagctgaaggtggaggcgccgcaagcgctgagagaaaatattctctttggaatgggaaatcctctgcttgacatctctgctgtagtggacaaagatttccttgataagtattctctgaaaccaaatgaccaaatcttggctgaagacaaacacaaggaactgtttgatgaacttgtgaaaaaattcaaagtcgaatatcatgctggtggctctacccagaattcaattaaagtggctcagtggatgattcaacagccacacaaagcagcaacattttttggatgcattgggatagataaatttggggagatcctgaagagaaaagctgctgaagcccatgtggatgctcattactacgagcagaatgagcagccaacaggaacttgtgctgcatgcatcactggtgacaacaggtccctcatagctaatcttgctgctgccaattgttataaaaaggaaaaacatcttgatctggagaaaaactggatgttggtagaaaaagcaagagtttgttatatagcaggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagaaagaaattattgataccaatggagctggagatgcatttgttggaggttttctgtctcaactggtctctgacaagcctctgactgaatgtatccgtgctggccactatgcagcaagcatcataattagacggactggctgcacctttcctgagaagccagacttccactga

SEQ ID NO: 2 - protein, 362 aa, Homo sapiens
MAAAEEEPKPKKLKVEAPQALRENILFGMGNPLLDISAVVDKDFLDKYSLKPNDQILAEDKHKELFDELVKKFKVEYHAGGSTQNSIKVAQWMIQQPHKAATFFGCIGIDKFGEILKRKAAEAHVDAHYYEQNEQPTGTCAACITGDNRSLIANLAAANCYKKEKHLDLEKNWMLVEKARVCYIAGFFLTVSPESVLKVAHHASENNRIFTLNLSAPFISQFYKESLMKVMPYVDILFGNETEAATFAREQGFETKDIKEIAKKTQALPKMNSKRQRIVIFTQGRDDTIMATESEVTAFAVLDQDQKEIIDTNGAGDAFVGGFLSQLVSDKPLTECIRAGHYAASIIIRRTGCTFPEKPDFH

SEQ ID NO: 3 - DNA, 1086 bp, Mus musculus
atggcagctgcggacgagcccaagcccaaaaagctcaaggtggaagcgccacaagcgctgagtgaaaatgtgctatttggaatggggaatcctcttcttgacatctctgctgtagtagacaaagatttccttgataagtattctctgaaaccaaatgaccagatcttggctgaagacaagcacaaggaactgtttgatgaacttgtgaaaaaattcaaagttgaatatcatgctggtggctctacgcagaattcaatgaaagtggctcagtggttgattcaggagccacacaaagcagcaacattctttggatgcattgggatagataagtttggggagatcctgaagcgtaaggctgctgacgcacatgtggatgctcattactatgagcagaacgagcagccaacaggaacttgtgctgcgtgtatcactggtggcaacaggtccctcgttgctaaccttgctgccgccaattgttacaagaaagagaagcaccttgacctggagcggaactgggtgttggtagagaaagccagagtttactacatagctggcttttttctcacagtctccccagagtcggtattgaaagtggctcgctatgctgccgagaacaacagggtcttcactttgaacctgtctgcaccgttcattagccagttcttcaaggaagccttgatggacgtcatgccttatgttgatatcctctttggaaatgagacggaagctgccacttttgctagagagcaaggctttgagactaaagacattaaagaaatagccaaaaaggcgcaggctcttccaaaggtgaactcgaagaggcagaggaccgtgatcttcacacaagggcgagatgacaccatagtggctgcagagaatgatgtcactgcttttcctgtcttggatcaaaaccaggaagagatcattgacaccaatggagctggagatgcatttgttggaggctttctgtctcaactggtctctgacaagcctctgactgagtgcatccgcgctggccactacgcagcaagcgtcatcattagacgaactggctgtacctttcccgagaagccagacttccactga

SEQ ID NO: 4 - protein, 361 aa, Mus musculus
MAAADEPKPKKLKVEAPQALSENVLFGMGNPLLDISAVVDKDFLDKYSLKPNDQILAEDKHKELFDELVKKFKVEYHAGGSTQNSMKVAQWLIQEPHKAATFFGCIGIDKFGEILKRKAADAHVDAHYYEQNEQPTGTCAACITGGNRSLVANLAAANCYKKEKHLDLERNWVLVEKARVYYIAGFFLTVSPESVLKVARYAAENNRVFTLNLSAPFISQFFKEALMDVMPYVDILFGNETEAATFAREQGFETKDIKEIAKKAQALPKVNSKRQRTVIFTQGRDDTIVAAENDVTAFPVLDQNQEEIIDTNGAGDAFVGGFLSQLVSDKPLTECIRAGHYAASVIIRRTGCTFPEKPDFH

SEQ ID NO: 5 - DNA, 1038 bp, Homo sapiens
atgacgtcagtcagagaaaatattctctttggaatgggaaatcctctgcttgacatctctgctgtagtggacaaagatttccttgataagtattctctgaaaccaaatgaccaaatcttggctgaagacaaacacaaggaactgtttgatgaacttgtgaaaaaattcaaagtcgaatatcatgctggtggctctacccagaattcaattaaagtggctcagtggatgattcaacagccacacaaagcagcaacattttttggatgcattgggatagataaatttggggagatcctgaagagaaaagctgctgaagcccatgtggatgctcattactacgagcagaatgagcagccaacaggaacttgtgctgcatgcatcactggtgacaacaggtccctcatagctaatcttgctgctgccaattgttataaaaaggaaaaacatcttgatctggagaaaaactggatgttggtagaaaaagcaagagtttgttatatagcaggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagaaagaaattattgataccaatggagctggagatgcatttgttggaggttttctgtctcaactggtctctgacaagcctctgactgaatgtatccgtgctggccactatgcagcaagcatcataattagacggactggctgcacctttcctgagaagccagacttccactga

SEQ ID NO: 6 - protein, 345 aa, Homo sapiens
MTSVRENILFGMGNPLLDISAVVDKDFLDKYSLKPNDQILAEDKHKELFDELVKKFKVEYHAGGSTQNSIKVAQWMIQQPHKAATFFGCIGIDKFGEILKRKAAEAHVDAHYYEQNEQPTGTCAACITGDNRSLIANLAAANCYKKEKHLDLEKNWMLVEKARVCYIAGFFLTVSPESVLKVAHHASENNRIFTLNLSAPFISQFYKESLMKVMPYVDILFGNETEAATFAREQGFETKDIKEIAKKTQALPKMNSKRQRIVIFTQGRDDTIMATESEVTAFAVLDQDQKEIIDTNGAGDAFVGGFLSQLVSDKPLTECIRAGHYAASIIIRRTGCTFPEKPDFH

SEQ ID NO: 7 - DNA, 1227 bp, Mus musculus
atgacgtccaccagtgaaaatgtgctatttggaatggggaatcctcttcttgacatctctgctgtagtagacaaagatttccttgataagtattctctgaaaccaaatgaccagatcttggctgaagacaagcacaaggaactgtttgatgaacttgtgaaaaaattcaaagttgaatatcatgctggtggctctacgcagaattcaatgaaagtggctcagtggttgattcaggagccacacaaagcagcaacattctttggatgcattgggatagataagtttggggagatcctgaagcgtaaggctgctgacgcacatgtggatgctcattactatgagcagaacgagcagccaacaggaacttgtgctgcgtgtatcactggtggcaacaggtccctcgttgctaaccttgctgccgccaattgttacaagaaagagaagcaccttgacctggagcggaactgggtgttggtagagaaagccagagtttactacatagctggcttttttctcacagtctccccagagtcggtattgaaagtggctcgctatgctgccgagaacaacagggtcttcactttgaacctgtctgcaccgttcattagccagttcttcaaggaagccttgatggacgtcatgccttatgttgatatcctctttggaaatgagacggaagctgccacttttgctagagagcaaggctttgagactaaagacattaaagaaatagccaaaaaggcgcaggctcttccaaaggtgaactcgaagaggcagaggaccgtgatcttcacacaagggcgagatgacaccatagtggctgcagagaatgatgtcactgcttttcctgtcttggatcaaaaccaggaagagatcattgacaccaatggagctggagatgcatttgttggaggctttctgtctcaactggtctctgacaagcctctgactgagtgcatccgcgctggccactacgcagcaagcgtcatcattagacgaactggctgtacctttcccgagaagccagacttccactgatggaagaaagaaaactcaggctgtggagtggagactgcagtgaccacatcctgagcgttcctccataagaaagaatagagttatcctctgtctttgcccaccatggttctcattagtaacttagagggctcagtgctacttctaggacctttagtctctgaaatctgggaaaaatgtttatttgcatag

SEQ ID NO: 8 - protein, 345 aa, Mus musculus
MTSTSENVLFGMGNPLLDISAVVDKDFLDKYSLKPNDQILAEDKHKELFDELVKKFKVEYHAGGSTQNSMKVAQWLIQEPHKAATFFGCIGIDKFGEILKRKAADAHVDAHYYEQNEQPTGTCAACITGGNRSLVANLAAANCYKKEKHLDLERNWVLVEKARVYYIAGFFLTVSPESVLKVARYAAENNRVFTLNLSAPFISQFFKEALMDVMPYVDILFGNETEAATFAREQGFETKDIKEIAKKAQALPKVNSKRQRTVIFTQGRDDTIVAAENDVTAFPVLDQNQEEIIDTNGAGDAFVGGFLSQLVSDKPLTECIRAGHYAASVIIRRTGCTFPEKPDFH

SEQ ID NO: 9 - DNA, 1101 bp, Homo sapiens
ggatccatggcagctgctgaggaggagccgaagcccaaaaagctgaaggtggaggcgccgcaagcgctgagagaaaatattctctttggaatgggaaatcctctgcttgacatctctgctgtagtggacaaagatttccttgataagtattctctgaaaccaaatgaccaaatcttggctgaagacaaacacaaggaactgtttgatgaacttgtgaaaaaattcaaagtcgaatatcatgctggtggctctacccagaattcaattaaagtggctcagtggatgattcaacagccacacaaagcagcaacattttttggatgcattgggatagataaatttggggagatcctgaagagaaaagctgctgaagcccatgtggatgctcattactacgagcagaatgagcagccaacaggaacttgtgctgcatgcatcactggtgacaacaggtccctcatagctaatcttgctgctgccaattgttataaaaaggaaaaacatcttgatctggagaaaaactggatgttggtagaaaaagcaagagtttgttatatagcaggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagaaagaaattattgataccaatggagctggagatgcatttgttggaggttttctgtctcaactggtctctgacaagcctctgactgaatgtatccgtgctggccactatgcagcaagcatcataattagacggactggctgcacctttcctgagaagccagacttccactgaaagctt

SEQ ID NO: 10 - DNA, 1167 bp, Mus musculus
ggatccactccgaggcgccggccagtgtgatggatttctgcagaatcgccctttaggtgcagtcatgacgtccaccagtgaaaatgtgctatttggaatggggaatcctcttcttgacatctctgctgtagtagacaaagatttccttgataagtattctctgaaaccaaatgaccagatcttggctgaagacaagcacaaggaactgtttgatgaacttgtgaaaaaattcaaagttgaatatcatgctggtggctctacgcagaattcaatgaaagtggctcagtggttgattcaggagccacacaaagcagcaacattctttggatgcattgggatagataagtttggggagatcctgaagcgtaaggctgctgacgcacatgtggatgctcattactatgagcagaacgagcagccaacaggaacttgtgctgcgtgtatcactggtggcaacaggtccctcgttgctaaccttgctgccgccaattgttacaagaaagagaagcaccttgacctggagcggaactgggtgttggtagagaaagccagagtttactacatagctggcttttttctcacagtctccccagagtcggtattgaaagtggctcgctatgctgccgagaacaacagggtcttcactttgaacctgtctgcaccgttcattagccagttcttcaaggaagccttgatggacgtcatgccttatgttgatatcctctttggaaatgagacggaagctgccacttttgctagagagcaaggctttgagactaaagacattaaagaaatagccaaaaaggcgcaggctcttccaaaggtgaactcgaagaggcagaggaccgtgatcttcacacaagggcgagatgacaccatagtggctgcagagaatgatgtcactgcttttcctgtcttggatcaaaaccaggaagagatcattgacaccaatggagctggagatgcatttgttggaggctttctgtctcaactggtctctgacaagcctctgactgagtgcatccgcgctggccactacgcagcaagcgtcatcattagacgaactggctgtacctttcccgagaagccagacttccactgatggaaagggcgaattccagcacactggcggccgttactagtggatccgagctcggtaccaagctt

(19) United States
(12) Patent Application Publication
(10) Pub. No.: US 2017/0219562 A1
(43) Pub. Date: Aug. 3, 2017

(54) EPIGENETIC COMPOUND SCREENING PLATFORM

(71) Applicant: Legacy Emanuel Hospital & Health Center, Portland, OR (US)

(72) Inventors: Detlev Boison, Portland, OR (US); Ursula Susan Sandau, Beaverton, OR (US)

(73) Assignee: Legacy Emanuel Hospital & Health Center, Portland, OR (US)

(21) Appl. No.: 15/418,427

(22) Filed: Jan. 27, 2017

Related U.S. Application Data
(60) Provisional application No. 62/289,845, filed on Feb. 1, 2016.

(51) Int. Cl. G01N 33/50; C12Q 1/68; C12N 5/071

(52) U.S. Cl. CPC G01N 33/5008; C12N 5/0686; C12Y 207/0102; C12Q 1/6876; G01N 2500/10; G01N 2500/04; G01N 2333/91017; C12Q 2600/154

(57) ABSTRACT

Epigenetic compound screening platform, including methods and cell lines. In an exemplary screening method, ADK-null, ADK-L, and ADK-S cell lines may be selected. The ADK-null cell line may express no ADK protein. The ADK-L cell line may express only the long (L), nuclear isoform of a mammalian ADK protein from an exogenous construct. The ADK-S cell line may express only the short (S), cytoplasmic isoform of a mammalian ADK protein from an exogenous construct. Each of the cell lines may be exposed to the same test compound. A level of DNA or histone methylation, or DNA or histone methyltransferase activity for each of the exposed cell lines may be measured. The level for each exposed cell line may be compared to a corresponding level measured without exposure to the test compound, to determine whether the test compound affects DNA or histone methylation, or DNA or histone methyltransferase activity, in any of the cell lines.

### CROSS-REFERENCE TO PRIORITY APPLICATION

This application is based upon and claims the benefit under 35 U.S.C. §119(e) of U.S. Provisional Patent Application Ser. No. 62/289,845, filed Feb. 1, 2016, which is incorporated herein by reference in its entirety for all purposes.

### INTRODUCTION

Environmental factors can produce stable changes to the chemical structure and/or packaging of DNA within a cell. These changes are described as epigenetic, rather than genetic, when they do not alter the primary sequence of the DNA. Epigenetic alterations can include DNA methylation and histone modification, among others. DNA methylation in mammals occurs at the 5 position of cytosine, typically at CpG dinucleotides, to produce 5-methylcytosine, and generally reduces transcription of associated genes. Histone modification can include methylation, phosphorylation, acetylation, ubiquitination, and sumoylation, among others, of one or more histone proteins, namely, histone H1/H5, H2A, H2B, H3, and/or H4, and can activate or repress associated genes.

Changes to the pattern of DNA methylation or histone modification can determine the progression of various chronic conditions, such as epilepsy, Alzheimer's disease, Parkinson's disease, schizophrenia, and cancer. Drugs that affect DNA methylation or histone modification are needed to treat these conditions.

### SUMMARY

The present disclosure provides an epigenetic compound screening platform, including methods and cell lines. In an exemplary screening method, ADK-null, ADK-L, and ADK-S cell lines may be selected. The ADK-null cell line may express no adenosine kinase (ADK) protein. The ADK-L cell line may express only the long (L), nuclear isoform of a mammalian ADK protein from an exogenous construct. The ADK-S cell line may express only the short (S), cytoplasmic isoform of a mammalian ADK protein from an exogenous construct. Each of the cell lines may be exposed to the same test compound. A level of DNA or histone methylation, or DNA or histone methyltransferase activity, for each of the exposed cell lines may be measured. The level for each exposed cell line may be compared to a corresponding level measured without exposure to the test compound, to determine whether the test compound affects DNA or histone methylation, or DNA or histone methyltransferase activity, in any of the cell lines.

### BRIEF DESCRIPTION OF THE DRAWINGS

FIG. 1 is a flowchart illustrating an exemplary screening method to identify compounds that alter DNA methylation, in accordance with aspects of the present disclosure.

FIG. 2 is a schematic representation of exemplary adenosine kinase (ADK) expression constructs that were integrated into the genome of ADK-null cells to generate stable, isoform-specific ADK cell lines, in accordance with aspects of the present disclosure.

FIG. 3 is an image of a western blot detecting expression of ADK long (L) and short (S) protein isoforms, and alpha-tubulin, in four different BHK cell lines: parental (WT), ADK-null, ADK-L, and ADK-S. Recombinant ADK-S protein (rADK-S) was used as a positive control.

FIG. 4 is a bar graph plotting the mean level of ADK expression detected by western blot analysis of each of the four BHK cell lines of FIG. 3, as assessed by densitometry of western blot images.

FIG. 5 is a series of images of each of the ADK-null, ADK-L, and ADK-S cell lines of FIG. 3 detected by confocal microscopy through differential interference contrast (DIC), fluorescence of a DAPI nuclear stain, or ADK immunofluorescence.

FIG. 6 is a bar graph plotting the amount of 5-methylcytosine detected in genomic DNA isolated from each of the ADK-null, ADK-L, and ADK-S cell lines of FIG. 3, with each amount normalized with respect to the amount detected for the ADK-null cell line.

FIG. 7 is a bar graph plotting the amount of 5-methylcytosine detected in genomic DNA isolated from the ADK-L cell line of FIG. 3 in the absence or presence of various ADK inhibitors, with vehicle-treated ADK-null cells as a control, and with each amount normalized with respect to the amount detected for vehicle-treated ADK-null cells.

FIG. 8 is a pair of bar graphs plotting the amount of 5-methylcytosine detected in genomic DNA isolated from the ADK-null cell line (Panel A) and the ADK-S cell line (Panel B) of FIG. 3, in the absence or presence of various ADK inhibitors, with each amount normalized with respect to the amount detected for vehicle-treated ADK-null cells.

FIG. 9 is a pair of bar graphs plotting the amount of 5-methylcytosine detected in genomic DNA isolated from the ADK-L cell line (Panel A) and the ADK-S cell line (Panel B) of FIG. 3, in the absence or presence of carbamazepine, and with vehicle-treated ADK-null cells as a control.

### DETAILED DESCRIPTION

The present disclosure provides an epigenetic compound screening platform, including methods and cell lines. In an exemplary screening method, ADK-null, ADK-L, and ADK-S cell lines may be selected. The ADK-null cell line may express no ADK protein. The ADK-L cell line may express only the long (L), nuclear isoform of a mammalian ADK protein from an exogenous construct. The ADK-S cell line may express only the short (S), cytoplasmic isoform of a mammalian ADK protein from an exogenous construct. Each of the cell lines may be exposed to the same test compound. A level of DNA or histone methylation (or other histone modification), or DNA or histone methyltransferase /modification activity, for each of the exposed cell lines may be measured. The level for each exposed cell line may be compared to a corresponding level measured without exposure to the test compound, to determine whether the test compound affects DNA or histone methylation/modification, or DNA or histone methyltransferase /modification activity, in any of the cell lines.

Compound screening may be performed with a single cell line, or with two or more cell lines, which may have different levels of DNA and/or histone methylation before exposure to a test compound. In some embodiments, compound screening may be performed with a cell line expressing a nuclear form of adenosine kinase (e.g., the long isoform of ADK) and at least substantially no cytoplasmic form of adenosine kinase (resulting in a DNA/histone hypermethylated cell line), and optionally, another cell line expressing a cytoplasmic form of adenosine kinase (e.g., the short isoform of ADK) and at least substantially no nuclear form of adenosine kinase (resulting in a cell line with intermediate DNA/histone methylation levels) and/or another cell line expressing substantially no functional adenosine kinase (resulting in a DNA/histone hypomethylated cell line).

The progression of chronic conditions, such as epilepsy, Parkinson's disease, Alzheimer's disease, and possibly cancer, can be determined by DNA/histone methylation. Compounds able to maintain and/or restore normal DNA/histone methylation can prevent disease progression and possibly cure neurodegenerative conditions. The cell-based screening platform disclosed herein can fast-track the identification of drugs for preventing disease-associated changes in DNA/histone methylation.

In some embodiments, the epigenetic screening platform may utilize different forms of adenosine kinase (ADK). The mammalian ADK protein has been shown to be expressed from the same gene as a long isoform and a short isoform that differ in length by about twenty amino acids. The long isoform contains a nuclear localization signal absent from the short isoform. Accordingly, the long isoform may be described as a nuclear form of ADK, while the short isoform may be described as a cytoplasmic form of ADK, because a greater proportion of the long isoform than the short isoform accumulates in the nucleus. Each form of ADK expressed by the cell lines of the present disclosure may be structurally identical to, or different from, a corresponding natural isoform. Therefore, a nuclear (or cytoplasmic) form of ADK expressed by a cell line may be longer or shorter than, or the same length as, the corresponding long or short isoform. Also, each form of ADK may be expressed from the endogenous ADK gene (e.g., an engineered, mutant ADK gene specifically expressing a long or short isoform, but not both), or expressed from an exogenous ADK expression construct.

The natural isoforms of ADK have different physiological roles. The short (cytoplasmic) isoform of ADK may control the tissue tone of adenosine and thereby the degree of adenosine receptor activation. The short isoform thus may be responsible for therapeutic benefits of adenosine but also for major side effects of ADK inhibitors. In contrast, the long (nuclear) isoform of ADK may have more control over the epigenetic functions of ADK, relative to the short isoform. Past drug development efforts involving ADK as a target were abandoned due to side effects, which may be related to inhibition of the short isoform of ADK. Inhibitors of ADK with higher specificity for the long isoform over the short isoform can capitalize on the beneficial epigenetic effects of ADK inhibitors, while avoiding side effects associated with an elevated tissue tone of adenosine produced by inhibition of the short isoform.

### I. OVERVIEW OF AN EXEMPLARY EPIGENETIC SCREENING PLATFORM

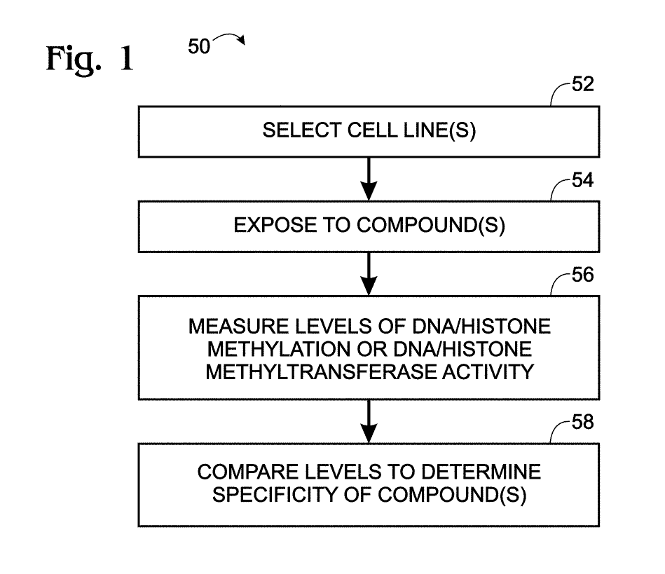

FIG. 1 shows a flowchart for an exemplary method 50 of epigenetic screening. The steps shown may be performed in any suitable order and combination, and may be modified with any other suitable aspects of the present disclosure. Each of the steps of method 50 may be performed manually, robotically, with a computer, or a combination thereof. The method may, in some cases, be configured for high-throughput screening of a library of compounds.

At least one cell line may be selected, indicated at 52. Each cell line that is selected may be created (e.g., by introducing genetic material into a parental cell line) or acquired (e.g., received from a repository, laboratory, commercial enterprise, etc.). Exemplary approaches for creating a cell line introduce a nucleic acid construct into cells as a precipitate (e.g., a calcium phosphate precipitate), by lipofection or electroporation, on projectiles, via infection with virus, or the like. The cell line may be an established (immortalized) cell line that can proliferate indefinitely in culture, or a stem cell line or derivative thereof. The cell line may be an adherent cell line that grows while attached to a substrate, a suspension cell line that can grow in suspension, or both. In some embodiments, each cell line is clonal, that is, produced by proliferation of a single progenitor cell.

Each cell line may originate from any suitable species, such as a vertebrate (e.g., mammalian) species. Exemplary mammalian cell lines are of primate (e.g., human), rodent (e.g., murine or hamster), bovine, canine, equine, feline, ovine, or porcine origin, among others. The cell line alternatively may originate from a non-vertebrate species, which may be eukaryotic or prokaryotic, among others. For example, the cell line may be composed of bacterial cells, plant cells, insect cells, yeast cells, or the like.

Each cell line may contain and/or be exposed to at least one nucleic acid construct, which may or may not become integrated into the genome of the cell line. The nucleic acid construct may include an inactivating construct and/or an expression construct.

The inactivating construct may be any exogenous polynucleotide that at least reduces or eliminates expression of an endogenous ADK gene. As a result, one of the cell lines may be an ADK-null cell line (interchangeably called an ADK-knockout cell line) expressing no ADK protein that is full-length, functional, and/or detectable. Expression may be reduced or eliminated by mutating a coding sequence, promoter, splice junction, polyadenylation signal, and/or the like of the gene. Mutation may occur by homologous recombination, or editing (e.g., via the CRISPR technique), among others. The inactivating construct may or may not be isoform-specific. For example, the inactivating construct may at least reduce or eliminate expression of both the long and the short isoforms of ADK, or may at least reduce or eliminate expression of the long isoform specifically, or the short isoform specifically. An inactivating construct may be integrated into the gene itself or function at a distance, such as by expression of a guide RNA for CRISPR editing, or expression of an interfering RNA that reduces expression of the gene, among others.

The expression construct may be any exogenous polynucleotide that causes expression of a protein of interest. An expression construct may include a coding sequence for the protein, an operatively linked promoter and other control sequences to drive and regulate expression, and the like. In exemplary embodiments, the expression construct encodes the long, nuclear isoform of ADK (ADK-L) or the short, cytoplasmic form of a mammalian ADK. The long isoform of ADK is present at a higher concentration in the nucleus than the cytoplasm, while the reverse holds for the short isoform of ADK.

The long (L) and short (S) isoforms of ADK are abbreviated throughout the present disclosure as ADK-L and ADK-S, respectively. The ADK-L and ADK-S isoforms from a given species are produced by alternative splicing of a primary ADK transcript, which generates ADK-L and ADK-S mRNAs that encode the corresponding ADK-L and ADK-S proteins. The L and S protein isoforms expressed from a given endogenous ADK gene have different amino terminal sequences. For example, the first 21 (human) or 20 (mouse) amino acids of ADK-L contain a nuclear localization signal (NLS). These amino acids are replaced by four amino acids in the S isoform (human or mouse), which eliminates the nuclear localization signal and shortens the protein by 17 amino acids. Accordingly, the L isoform is relatively more nuclear, while the S isoform more cytoplasmic. Besides this difference at the amino terminus, the ADK-L and ADK-S proteins encoded by a given endogenous ADK gene may be identical to one another in amino acid sequence.

The ADK-L coding sequence may originate from any suitable vertebrate species, such as a mammalian species. An exemplary human ADK-L coding sequence is presented as SEQ ID NO:1, which encodes an ADK-L protein having SEQ ID NO:2. An exemplary mouse ADK-L coding sequence is presented as SEQ ID NO:3, which encodes an ADK-L protein having SEQ ID NO:4. The human and mouse ADK-L proteins of SEQ ID NO:2 and SEQ ID NO:4 are highly homologous, exhibiting 91% amino acid sequence identity. Accordingly, the ADK-L protein encoded by the expression construct of an ADK-L cell line may, for example, have at least 80%, 90%, 95%, 97%, 98%, 99%, or 100% sequence identity with SEQ ID NO:2 or SEQ ID NO:4. The ADK-L protein also or alternatively may have a length (as measured by the total number of amino acids) that is at least 90%, 95%, 98%, or 99% of (or the same as) the length of SEQ ID NO:2 or SEQ ID NO:4.

The ADK-S coding sequence also may originate from any suitable vertebrate species, such as a mammalian species. An exemplary human ADK-S coding sequence is presented as SEQ ID NO:5, which encodes an ADK-S protein having SEQ ID NO:6. An exemplary mouse ADK-S coding sequence is presented as SEQ ID NO:7, which encodes an ADK-S protein having SEQ ID NO:8. The human and mouse ADK-S proteins of SEQ ID NO:6 and SEQ ID NO:8 are highly homologous, exhibiting 91% amino acid identity. Accordingly, the ADK-S protein encoded by the expression construct of an ADK-S cell line may have at least 80%, 90%, 95%, 97%, 98%, 99%, or 100% sequence identity with SEQ ID NO:6 or SEQ ID NO:8. The ADK-S protein also or alternatively may have a length (as measured by the total number of amino acids) that is at least 90%, 95%, 98%, or 99% (or the same as) the length of SEQ ID NO:6 or SEQ ID NO:8.

The L and S isoforms of ADK expressed by respective ADK-L and ADK-S cell lines may or may not originate from the same species. If the two isoforms originate from the same species, they may have identical sequences, except at their respective amino termini, as described above. Alternatively, the L and S isoforms may represent different alleles of the ADK gene from the same species, and thus may have one or more other amino acid differences outside of their differing amino termini.

Each cell line may be exposed to one or more compounds, indicated at 54. Each compound may be described as a “test compound” for which an effect, if any, on DNA or histone methylation (or DNA or histone methyltransferase activity) in the cell line is being tested. The compound may be a substance having a single, defined chemical formula. The compound may have any suitable molecular weight, such as less than 10,000 Daltons. Exposure may, for example, be performed by introducing the compound into a liquid medium, such as a culture medium, that contacts cells of the cell line. The compound generally needs to enter the cells to act on ADK. In exemplary embodiments, sets of cells of the cell line may be distributed to an array of isolated compartments, such as wells, formed by at least one sample holder (such as a multi-well plate). Different compounds may be introduced into the compartment for testing on the sets of cells of the cell line therein. The cells of the cell line in at least one of the compartments may be exposed to no compound, as a control.

Levels of DNA or histone methylation, and/or DNA or histone methyltransferase activity may be measured, indicated at 56. A level may be measured with and without exposure to each test compound, for each cell line. The level may be measured after exposure of cells of a cell line to a compound for any suitable interval, such as at least 1, 2, 5, 10, or 30 minutes, at least 1, 2, 4, 8, or 16 hours, or at least 1, 2, 4, or 7 days, among others. A level of DNA/histone methylation or DNA/histone methyltransferase activity may be measured in intact cells or after lysing the cells. For example, the methylation or methyltransferase activity may be measured in a whole cell lysate, a cytosolic or nuclear lysate, or isolated (e.g., purified) genomic DNA and/or histones/nucleosomes, among others. The level of DNA/histone methylation or DNA/histone methyltransferase activity may be measured at a single time point after the start of exposure to a test compound, or at multiple time points in a kinetic assay.

Levels of DNA methylation may, for example, be measured in a ligand binding assay (e.g., an immunoassay, such an ELISA) performed with a 5-methylcytosine (5-mC) binding agent, such as an antibody to 5-mC or a methyl CpG binding domain (MBD) protein, among others. Genomic DNA isolated from cells exposed to a test compound may be attached to a surface (such as a surface inside a well of a multi-well plate). The immobilized DNA may be contacted with the 5-mC binding agent to allow binding to 5-mC moieties therein. After washing away unbound binding agent, the amount of agent bound (and thus the amount of 5-mC) may, for example, be measured with an enzyme associated with the binding agent (e.g., covalently attached or associated via noncovalent interaction). The enzyme may, for example, produce a detectable signal, such as emission of light, by catalyzing conversion of a substrate to a product.

Levels of histone methylation also may be measured in a ligand binding assay (e.g., an ELISA or other immunoassay), generally as described above for DNA methylation, but with a binding agent, such as an antibody, that specifically recognizes a methylated form of a histone. The binding agent may, for example, bind specifically to a particular histone (e.g., methylated histone H1/H5, H2A, H2B, H3, or H4), optionally only when the histone has a particular residue methylated (e.g., methylation of lysine-9 or lysine-27 in histone H3).

The level of DNA methylation also or alternatively may be measured by a bisulfite sequencing protocol. Bisulfite treatment of DNA converts unmethylated cytosine residues to uracil, but leaves 5-mC residues unaffected. Sequencing bisulfite-treated DNA distinguishes unmethylated C residues in the genome that have been converted to T, from methylated C residues that are still read as C. In some embodiments, reduced representation bisulfite sequencing (RRBS) may be performed. RRBS is a high-throughput approach to directly sequence the DNA and thus determine sites of altered DNA methylation.

The level of DNA or histone methyltransferase activity also or alternatively may be measured. Inhibition of DNA methyltransferases (DNMTs, such as DNMT1, DNMT3a, and/or DNMT3b) or histone methyltransferases may be informative as a more direct effect of ADK inhibition. Changes in DNA or histone methylation are downstream of the DNA or histone methyltransferases. A DNMT or histone methyltransferase assay may, for example, be performed with a DNA or histone substrate immobilized on a surface (e.g., in a well of a multi-well plate). The histone substrate may, for example, be a single type of histone (H1, H2A, H2B, H3, or H4), a histone octamer (e.g., containing two copies of histone H2A, H2B, H3, and H4), a nucleosome, chromatin, or the like. DNMT activity present in a lysate, such as a nuclear lysate, transfers a methyl group to cytosine residues of the immobilized DNA, which causes a 5-mC binding agent (e.g., a 5-mC antibody or an MBD protein) to bind to the methylated DNA. An enzyme linked to or otherwise associated with the 5-mC binding agent can generate a detectable signal from a suitable substrate (e.g., by catalyzing conversion of the substrate to a colored product). Similarly, histone methyltransferase activity present in a lysate transfers methyl groups to histone, which can, for example, be detected by ELISA using an antibody that binds specifically to a methylated form of a histone, as described above.

Compounds with any suitable desired property may be identified. For example, one or more compounds that reduce the level of DNA/histone methylation and/or DNMT or histone methyltransferase activity in a cell line may be identified. In some embodiments, the compounds may reduce the level in a cell line that is DNA/histone hypermethylated (pre-exposure) (such as an ADK-L cell line). Alternatively, or in addition, one or more compounds that increase the level of DNA/histone methylation and/or DNMT or histone methyltransferase activity in a cell line may be identified. In some embodiments, the compounds may increase the level in a cell line that is DNA/histone hypomethylated (pre-exposure) (such as an ADK-null cell line).

The levels measured may be compared to determine a specificity, if any, of each compound for one or more of the cell lines, indicated at 58. The specificity may be determined by comparing levels measured in step 54, for each cell line in the presence and absence of the test compound, and/or between or among cell lines each exposed to the same test compound. For example, a level measured from an ADK-L cell line may be compared with a level measured from an ADK-S cell line and, optionally, an ADK-null cell line. A compound with the desired property may selectively affect (e.g., reduce) DNA/histone methylation or DNA/histone methyltransferase activity in the ADK-L cell line relative to the ADK-S cell line, with little or no effect on the ADK-null cell line. In some examples, a compound that is not an inhibitor of ADK may selectively reduce DNA/histone methylation of a hypermethylated cell line (e.g., the ADK-L cell line) relative to a control cell line with normal or below-normal levels of DNA/histone methylation (e.g., the ADK-S cell line). In other examples, a compound with the desired property may increase DNA/histone methylation in a cell line. The compound may selectively increase DNA/histone methylation of a hypomethylated cell line (e.g., ADK-null) relative to a normally methylated and/or hypermethylated cell line (e.g., ADK-L and/or ADK-S).

At least one of the compounds may be a direct or indirect inhibitor of ADK enzyme activity and/or DNA (or histone) methylation or DNA (or histone) methyltransferase (or demethylase) activity. A direct inhibitor of ADK may be a reversible or irreversible inhibitor of ADK enzyme activity. The direct inhibitor binds to ADK and may be a competitive, noncompetitive/mixed, or uncompetitive inhibitor. In contrast, an indirect inhibitor of ADK does not bind directly to ADK. Similarly, a direct inhibitor of DNA or histone methyltransferase may bind to a DNA methyltransferase enzyme or a histone methyltransferase enzyme.

### II. EXAMPLES

The following examples describe further aspects of exemplary epigenetic screening platforms. These examples are intended for illustration and should not define or limit the entire scope of the present disclosure.

### Example 1

### Exemplary Development of a Screening Platform

Stable cell lines, such as derivatives of the baby hamster kidney (BHK) cell line, may be engineered that either express the short, cytoplasmic isoform or the long, nuclear isoform of ADK (and that lack any endogenous expression of functional ADK). These cell lines have defined levels of DNA hypermethylation. The cell lines may be incorporated into a screening platform to identify isoform-selective ADK inhibitors and to screen for compounds that affect (i.e., reduce or increase) DNA or histone methylation, and/or DNA or histone methyltransferase activity. The screening platform could be utilized for high-throughput screening assays.

Experiments demonstrate that DNA methylation drives disease progression in epilepsy, (2) adenosine regulates DNA methylation, (3) therapeutic adenosine augmentation restores normal DNA methylation levels, and (4) thereby prevents disease progression in epilepsy. Importantly, adenosine kinase (ADK) is the key driver for increased DNA methylation; blockade of ADK therefore reduces DNA methylation status. A cell-based screening platform may identify compounds that reduce DNA methylation in ADK-overexpressing cultured cells.

An ADK-deficient BHK cell line (ADK-null) has been developed. The ADK-null cells have a low DNA methylation status. The transient introduction of the short, cytoplasmic isoform of ADK (ADK-S), and even more so the introduction of the long, nuclear isoform of ADK (ADK-L), lead to increased DNA methylation levels. A neomycin or similar selection marker (e.g., puromycin, etc.) may be added to the gene expression constructs for ADK-S and ADK-L. The ADK-null cells may be transduced with the expression constructs for ADK-S and ADK-L, and single clones may be selected for stable integration and expression of ADK-S and ADK-L. ADK expression may be quantified by Western Blot analysis and DNA methylation levels may be quantified by enzyme-linked immunosorbent assay (ELISA) of cell lysates. Three different cell lines may be obtained, as listed below in Table 1.

TABLE 1
Comparison of BHK cell lines

| Cell Line | ADK Isoform | Methylation Status |
|---|---|---|
| BHK | None | Hypomethylation of DNA (control) |
| ADK-null | | |
| BHK ADK-S | Short, cytoplasmic | Intermediate methylation of DNA |
| BHK ADK-L | Long, nuclear | Hypermethylation of DNA |

The screening platform may be validated with known ADK inhibitors. Cells may be treated with the ADK inhibitors 5-lodotubercidin and ABT-702, as well as a random control compound (e.g., valproate). Cells may be incubated for six hours with five different doses of each compound. Afterwards, 5-methylcytosine (5-mC) as marker for DNA methylation may be quantified in cell lysates via an immunoassay, such as an ELISA. The assays may be performed in triplicate and may yield a dose response profile with the expectation that ADK inhibitors, but not the control compound, lead to a reduction of DNA methylation. The assay can, for example, be performed in a 96-well format.

The screening platform disclosed herein may be utilized to characterize the properties of new ADK inhibitors. The new ADK inhibitors may have epigenetic effects (i.e., reduction of DNA methylation status), in addition to inhibition of ADK activity. The screening platform may allow identification of compounds that are specific for either the nuclear isoform or the cytoplasmic isoform of ADK. Drugs that are specific for the nuclear isoform may be extremely useful as “epigenetic drugs” that alter the epigenome without affecting adenosine receptor-dependent pathways. Compound screening may identify clinically tractable drugs that offer hope for a cure of progressive neurological conditions and neurodegenerative diseases.

The screening platform also may identify any compound that changes DNA methylation, irrespective of ADK inhibition. For example, if a compound from a library increases DNA methylation in the ADK-null cell line, the compound would likely act on DNA methylation, though through a mechanism independent of ADK.

Once a compound with an epigenetic effect has been identified, the next step may be to test the compound in an in vitro assay for inhibition of ADK enzyme activity. The in vitro assay for ADK activity may determine the IC50 of the drug.

After a compound with a desired effect on DNA methylation has been identified in vitro using the cell lines, additional validation/screening tests could be performed in an animal model. Application of the compound(s) to animals could be followed by DNA methylation assays, including any of the assays described herein.

### Example 2

### Characterization of ADK-Null, ADK-L, and ADK-S Cell Lines

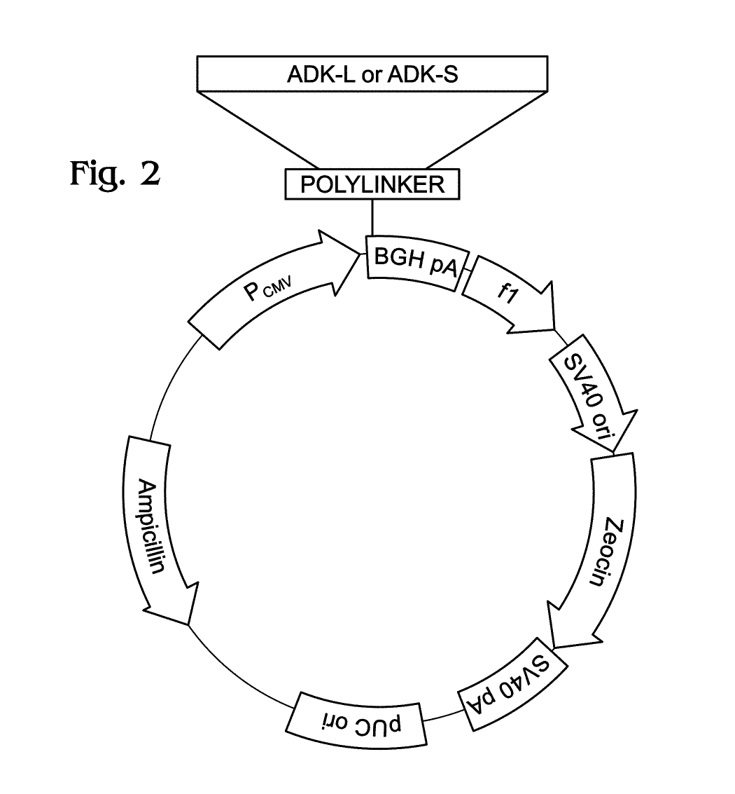

This example describes experiments characterizing exemplary BHK cell lines expressing no ADK (ADK-null), only the long (L) isoform of ADK (ADK-L), or only the short (S) isoform of ADK (ADK-S); see FIGS. 2-9.

FIG. 2 shows a schematic representation of two ADK expression constructs that were created and then integrated into the genome of a BHK ADK-null cell line to generate stable, ADK isoform-specific BHK cell lines, ADK-L and ADK-S, each derived as stable clones from the same parental ADK-null cell line. Each of the ADK expression constructs has the same backbone, pcDNA3.1/Zeo(−) (Invitrogen Life Technologies), and a different inserted sequence (encoding either a mammalian ADK-L protein or a mammalian ADK-S protein). Functional elements of the backbone include, in order, an empty expression module (Pcmv, polylinker, and BGH pA), a bacteriophage replication origin (f1), a zeocin expression module (SV40 promoter (SV40 ori), zeocin coding sequence, and SV40 polyadenylation sequence (SV 40 pA)), a bacterial replication origin (pUC ori), and an ampicillin resistance gene. The empty expression module includes a cytomegalovirus (CMV) promoter (P_{CMV}), a multiple cloning site (interchangeably termed a polylinker) into which each coding sequence is introduced, and a bovine growth hormone polyadenylation sequence (BGH pA).

Long (L) and short (S) isoform coding sequences for ADK from human and mouse, respectively, were obtained. Each coding sequence is flanked by a BamHI site upstream of the start codon (ATG), and a Hindil site downstream of the stop codon. The presence of the restriction enzyme sites enables directional insertion between corresponding upstream and downstream restriction enzyme sites in the polylinker of pcDNA3.1/Zeo(−), with preservation of both sites. The respective sequences into pcDNA3.1/Zeo(−), including the added BamHI and HindIII sites, are SEQ ID NO:9 for ADK-L and SEQ ID NO:10 for ADK-S. The human ADK-L coding sequence (for transcript variant 2) also is contained within NCBI Reference Sequence NM_006721.3. The mouse ADK-S coding sequence (for transcript variant 2) also is contained within NCBI Reference Sequence NM_001243041.1. (The designations “variant 1” and “variant 2” are reversed in mouse ADK relative to human ADK.) The size of each resulting ADK-L and ADK-S pcDNA3.1/Zeo(−) expression plasmid is 6.1 kb, and the amino acid sequences of the respective expressed human and mouse proteins are SEQ ID NO:2 and SEQ ID:8.

FIG. 3 shows an image of a western blot detecting expression of ADK long (L) and short (S) protein isoforms in four different BHK cell lines. Total protein lysates (5 μg) (lanes 1-4) and purified recombination ADK-S protein (lane 5) were resolved by polyacrylamide gel electrophoresis (PAGE) and then transferred to a membrane. The membrane was probed simultaneously with rabbit anti-ADK and mouse anti-α-tubulin primary antibodies, and then antibody binding was detected with a labeled secondary antibody by chemiluminescence. Detection of α-tubulin is performed as a gel loading control. The protein lysates were prepared from unmodified, progenitor (“wild type”) BHK cells (WT, lane 1); ADK-knockout BHK cells (ADK-null, lane 2); ADK-knockout BHK cells genetically modified to stably express the human ADK long isoform (ADK-L, lane 3); and ADK-knockout BHK cells genetically modified to stably express the mouse ADK short isoform (ADK-S, lane 4). Purified recombinant ADK short isoform (rADK-S) was run as a positive control (lane 5).

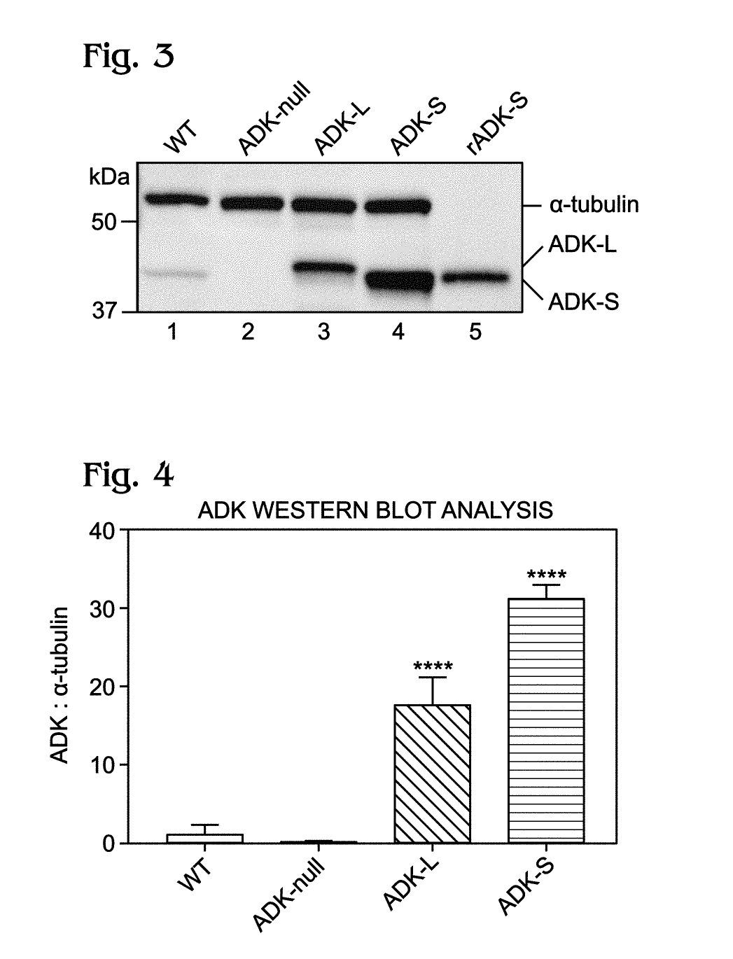

FIG. 4 shows a bar graph plotting the amount of ADK expression detected by western blot analysis of the four BHK cell lines of FIG. 3, as assessed by densitometry of western blot images obtained as in FIG. 3. The amount of ADK expression is plotted as a ratio of ADK protein to alpha-tubulin, with normalization of the ratio relative to unmodified progenitor BHK cells (WT). Data are represented as the mean±the standard error of the mean (SEM), with n=4 for the WT, ADK-null, and ADK-L cell lines, and n=3 for the ADK-S cell line. Statistical analysis is a one-way analysis of variance (One-way ANOVA) (F_{(3,11)}=165, p<0.0001) followed by Tukey's multiple comparisons post hoc test. The four asterisks (****) above the ADK-L and ADK-S bars indicate p<0.0001 versus each of the control cell lines (WT and ADK-null).

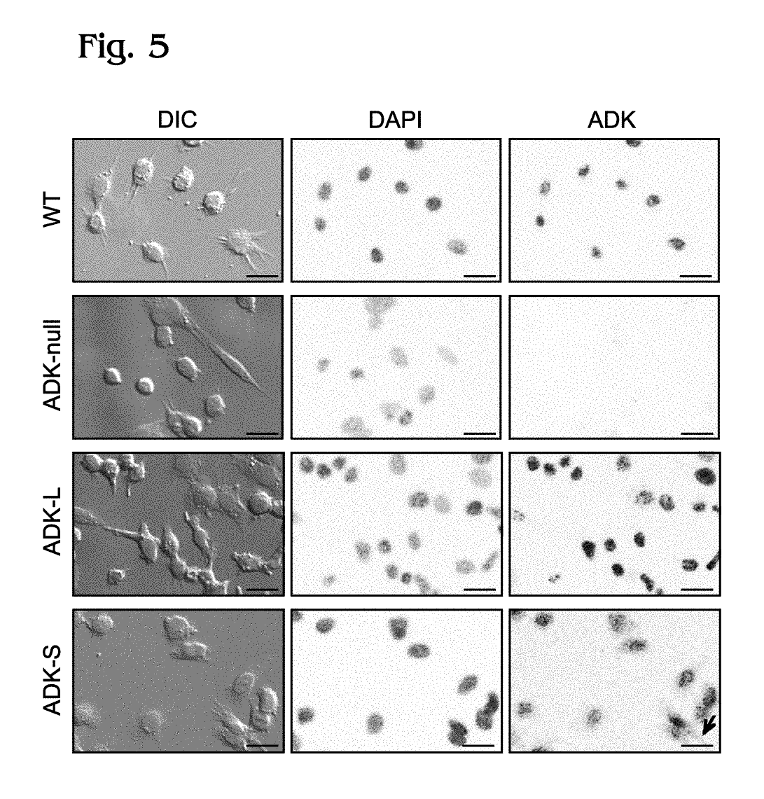

FIG. 5 shows a series of images of each of the cell lines of FIG. 3 detected by confocal microscopy through differential interference contrast (DIC), fluorescence of a DAPI nuclear stain, or ADK immunofluorescence. The DIC images in the left column show the complete cell shape including the nucleus and cytoplasm. The middle and right columns show z-stack projections of confocal microscopy images of the nuclear DAPI stain (middle) and ADK immunofluorescence (right). Fluorescence images of the middle column and of the right column were taken with identical settings within each column. ADK-null cells do not express any detectable ADK protein. ADK localization within ADK-L cells is confined to the nucleus. ADK localization within ADK-S cells is more diffuse and extends beyond the nucleus throughout the cytoplasm and into cell outgrowths (see arrow). The scale bar in the bottom right corner of each image represents 25 μm.

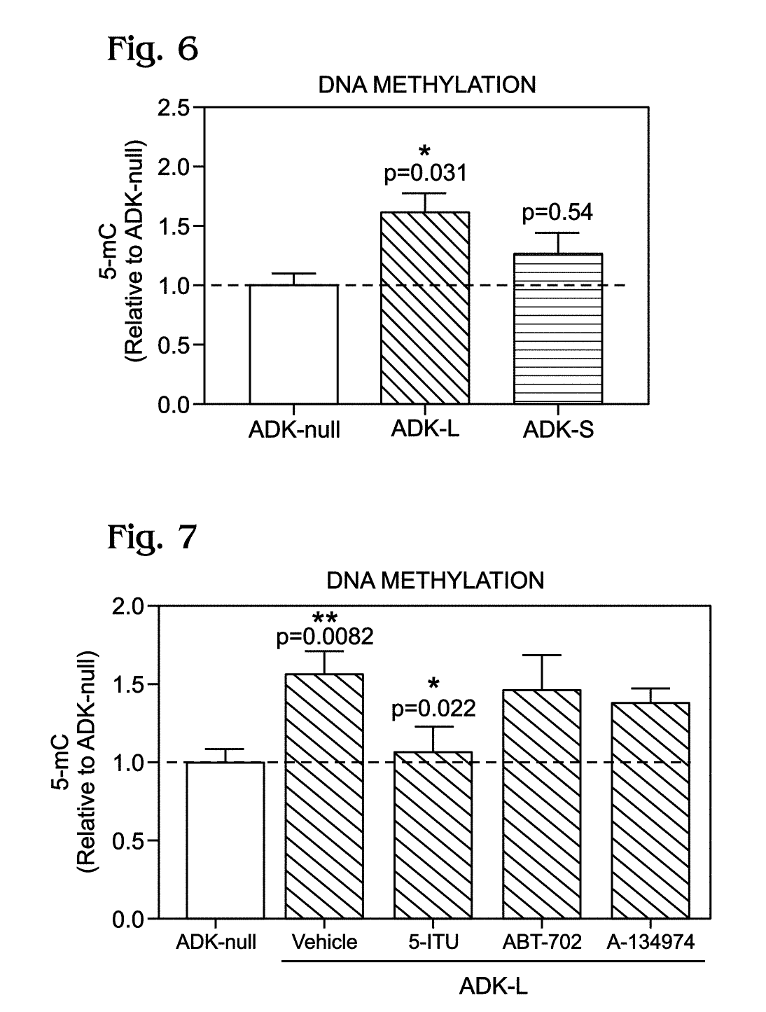

FIG. 6 shows a bar graph plotting the amount of 5-methylcytosine detected in genomic DNA isolated from each of the ADK-null, ADK-L, and ADK-S cell lines of FIG. 3, with each amount normalized with respect to the amount detected for the ADK-null cell line. Total genomic DNA was isolated from lysates of ADK-null cells (n=9), ADK-L cells (n=9), and ADK-S cells (n=9). 100 ng of purified genomic DNA was run on a 5-methylcytosine (5-mC) ELISA (Zymo Research Corp.) to quantify 5-methylcytosine. DNA methylation levels are significantly increased in ADK-L cells (p=0.031), compared to ADK-null cells. ADK-S cells do not have a significantly different level of DNA methylation from ADK-null cells (p=0.54). Data are represented as the mean±SEM. Statistical analysis is a One-way ANOVA (F_{(2,24)}=3.772, p=0.038) followed by Tukey's multiple comparisons post hoc test. The single asterisk (*) indicates p<0.05 versus ADK-null cells.

FIG. 7 is a bar graph plotting the amount of 5-methylcytosine detected in genomic DNA isolated from the ADK-L cell line of FIG. 3 in the absence or presence of various ADK inhibitors, with vehicle-treated ADK-null cells as a control, and with each amount normalized with respect to the amount detected for vehicle-treated ADK-null cells. ADK-L cells were treated with vehicle or the ADK inhibitor 5-iodotubercidin (5-ITU, 26 nM), ABT-702 (1.7 nM) or A-134974 (60 pM). The IC₅₀ for each ADK inhibitor was selected as the treatment dose. Total genomic DNA was isolated 24 hours after treatment and 100 ng of purified DNA was run on a 5-methylcytosine (5-mC) ELISA (Zymo Research Corp.) to quantify 5-methylcytosine. DNA methylation levels are significantly increased in vehicle-treated ADK-L cells (**, p=0.0082), compared to ADK-null cells. 5-ITU significantly decreases DNA methylation in ADK-L cells (*, p=0.022) versus vehicle alone. Neither ABT-702 nor A-134974 alters DNA methylation in ADK-L cells. Data are represented as the mean±SEM (n=8-9). Statistical analysis is a One-way ANOVA (F_{(4,37)}=2.95, p=0.033) followed by uncorrected Fisher's LSD post hoc test.

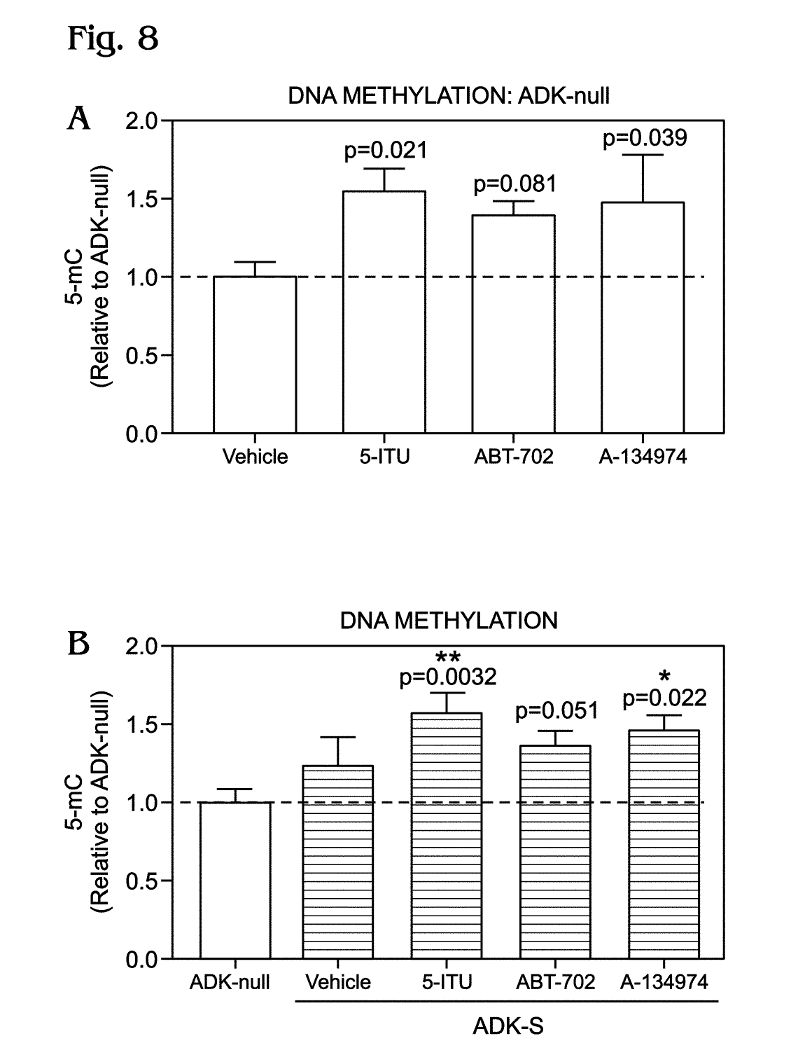

FIG. 8 is a pair of bar graphs plotting the amount of 5-methylcytosine detected in genomic DNA isolated from the ADK-null cell line (Panel A) and the ADK-S cell line (Panel B) of FIG. 3, in the absence or presence of various ADK inhibitors, with each amount normalized with respect to the amount detected for vehicle-treated ADK-null cells. ADK-null and ADK-S cells identify off target effects of ADK inhibitors on DNA methylation levels. ADK-null and ADK-S cells were treated with vehicle or the ADK inhibitor 5-lodotubercidin (5-ITU, 26 nM), ABT-702 (1.7 nM), or A-134974 (60 pM). The IC₅₀ for each ADK inhibitor was selected as the treatment dose. Total genomic DNA was isolated 24 hours after treatment, and 100 ng of purified DNA was run on a 5-methylcytosine (5-mC) ELISA (Zymo Research Corp) to determine the amount of 5-methylcytosine. Panel A shows a trend towards increased DNA methylation levels in ADK-null cells treated with commercial ADK inhibitors (F_{(3,17)}=3.16, p=0.052), compared to vehicle-treated ADK-null cells. This demonstrates off-target effects of commercially available ADK inhibitors. Likewise, Panel B shows DNA methylation levels are significantly increased in 5-ITU (**p=0.0032) and A-134974 (*p=0.022) treated ADK-S cells, compared to vehicle-treated ADK-S cells. Data are represented as the mean±SEM (n=4-9). Statistical analysis is a One-way ANOVA followed by uncorrected Fisher's LSD post hoc test.

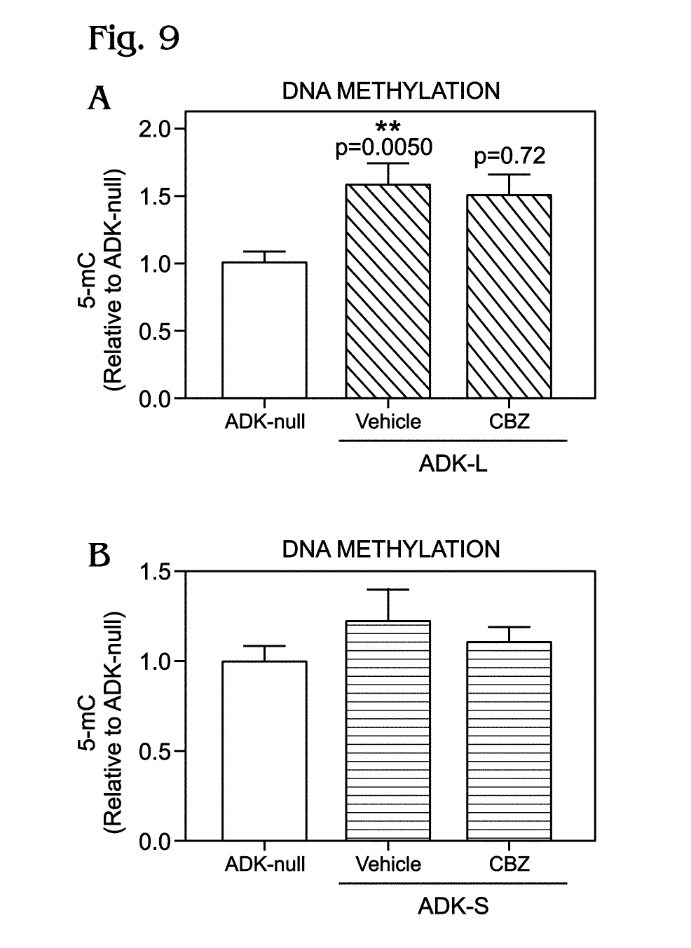

FIG. 9 is a pair of bar graphs plotting the amount of 5-methylcytosine detected in genomic DNA isolated from the ADK-L cell line (Panel A) and the ADK-S cell line (Panel B) of FIG. 3, in the absence or presence of carbamazepine, with vehicle-treated ADK-null cells as a control. The data show isoform-specific effects of carbamazepine on DNA methylation levels in ADK-L and ADK-S cells. Carbamazepine (CBZ) is a voltage-gated sodium channel blocker prescribed as a standard anti-epileptic drug, a mood stabilizer, and as therapy for neuropathic pain. CBZ does not interact with ADK, but has reported effects on DNA methylation (Asai et al, International Journal of Neuropsychopharmacology (2013), 16, 2285-2294.) ADK-L cells or ADK-S cells were treated with vehicle or CBZ (24.5 nM). The IC₅₀ of CBZ was selected as the treatment dose. Total genomic DNA was isolated 24 hours after treatment, and 100 ng of purified DNA was run on a 5-methylcytosine (5-mC) ELISA (Zymo Research Corp) to determine the amount of 5-methylcytosine. In Panel A, DNA methylation levels are significantly increased in vehicle (**p=0.0050) compared to ADK-null cells. CBZ does not decrease DNA methylation in ADK-L cells (p=0.72). In Panel B, DNA methylation levels are not significantly different in vehicle-treated or CBZ-treated ADK-S cells compared to ADK-null cells. Data are represented as the mean±SEM (n=8-9). Statistical analysis is a One-way ANOVA for Panel A (F_{(2,23)}=5.67, p=0.0099) followed by uncorrected Fisher's LSD post hoc test and Kruskal-Wallis test for Panel B (Kruskal-Wallis statistic=1.11, p=0.57).

### Example 3

### Selected Embodiments A

This example presents selected embodiments of the present disclosure as a series of indexed paragraphs.

Paragraph 1. A method of compound screening, the method comprising: (A) obtaining a cell line expressing a nuclear form of adenosine kinase and at least substantially no cytoplasmic form of adenosine kinase; (B) exposing the cell line to a compound; and (C) measuring a level of DNA or histone methylation, or DNA or histone methyltransferase activity, for the cell line.

Paragraph 2. The method of paragraph 1, wherein the compound is a direct or indirect inhibitor of DNA or histone methylation.

Paragraph 3. The method of paragraph 1 or 2, wherein the compound is an inhibitor of adenosine kinase.

Paragraph 4. A method of compound screening, the method comprising: (A) obtaining a first cell line expressing a nuclear form of adenosine kinase and at least substantially no cytoplasmic form of adenosine kinase, and a second cell line expressing a cytoplasmic form of adenosine kinase and at least substantially no nuclear form of adenosine kinase; (B) exposing each cell line to a compound; and (C) measuring a level of DNA or histone methylation, or DNA or histone methyltransferase activity, for each cell line.

Paragraph 5. The method of paragraph 4, wherein the compound is a direct or indirect inhibitor of DNA or histone methylation.

Paragraph 6. The method of paragraph 4 or 5, wherein the compound is an inhibitor of adenosine kinase.

Paragraph 7. The method of any of paragraphs 4 to 6, wherein each cell line expresses a form of adenosine kinase from an exogenous construct integrated into the genome of the cell line.

Paragraph 8. The method of any of paragraphs 4 to 7, wherein the step of measuring a level is performed with a binding reagent that binds specifically to methylated DNA or a methylated histone.

Paragraph 9. The method of paragraph 8, wherein the binding reagent is an antibody that binds to 5-methylcytosine, or that binds to a methylated form of a histone selected from the group consisting of histones H1/H5, H2A, H2B, H3A, H3B, and H4.

Paragraph 10. The method of any of paragraphs 4 to 9, wherein the step of measuring is performed with DNA or histones immobilized on a substrate.

Paragraph 11. The method of any of paragraphs 4 to 10, wherein the step of measuring includes a step of measuring DNA methyltransferase activity or histone methyltransferase activity present in a cell lysate.

Paragraph 12. The method of any of paragraphs 4 to 7, wherein the step of measuring includes a step of performing a sequencing reaction.

Paragraph 13. The method of any of paragraphs 4 to 12, further comprising a step of determining a specificity, if any, of the compound for the first cell line relative to the second cell line.

Paragraph 14. A system for screening drugs, comprising: (A) a first cell line stably expressing an exogenous, nuclear form of adenosine kinase; and (B) a second cell line stably expressing an exogenous, cytoplasmic form of adenosine kinase.

Paragraph 15. The system of paragraph 14, wherein each cell line is deficient for endogenous adenosine kinase.

Paragraph 16. The system of paragraph 14 or 15, wherein each cell line is a baby hamster kidney cell line.

Paragraph 17. The system of any of paragraphs 14 to 16, further comprising a third cell line deficient for endogenous adenosine kinase and expressing neither a nuclear form nor a cytoplasmic form of adenosine kinase.

### Example 4

### Selected Embodiments B

This example present additional selected embodiments of the present disclosure as a series of indexed paragraphs.

Paragraph 1. A screening method to identify compounds that alter DNA methylation, the method comprising, the method comprising: (A) selecting an ADK-null cell line, an ADK-L cell line, and an ADK-S cell line, wherein each copy of an endogenous adenosine kinase (ADK) gene has been inactivated in each of the cell lines, wherein the ADK-null cell line does not express any ADK protein, wherein the ADK-L cell line expresses the long (L), nuclear isoform of a mammalian ADK protein from an exogenous construct, and wherein the ADK-S cell line expresses the short (S), cytoplasmic isoform of a mammalian ADK protein from an exogenous construct; (B) exposing each of the cell lines to the same test compound; (C) measuring a level of DNA or histone methylation, or DNA or histone methyltransferase activity, for each of the exposed cell lines; and (D) comparing the level for each exposed cell line to a corresponding level measured without exposure to the test compound, to determine whether the test compound affects DNA or histone methylation, or DNA or histone methyltransferase activity in any of the cell lines.

Paragraph 2. The method of paragraph 1, wherein the step of comparing includes a step of determining whether exposure to the test compound increases DNA or histone methylation, or DNA or histone methyltransferase activity, in the ADK-null cell line.

Paragraph 3. The method of paragraph 1 or paragraph 2, wherein the step of comparing includes a step of determining whether exposure to the test compound decreases DNA or histone methylation, or DNA or histone methyltransferase activity, in the ADK-L cell line and in the ADK-S cell line.

Paragraph 4. The method of paragraph 3, wherein the step of comparing includes a step of determining a specificity, if any, of the test compound for reducing DNA or histone methylation, or DNA or histone methyltransferase activity, in the ADK-L cell line relative to the ADK-S cell line.

Paragraph 5. The method of any of paragraphs 1 to 4, wherein each of the ADK-L and ADK-S cell lines is a clone produced by transfection of the ADK-null cell line with an ADK-L or ADK-S expression construct.

Paragraph 6. The method of any of paragraphs 1 to 5, wherein the step of comparing includes a step of comparing the level for the ADK-L cell line with the level for the ADK-null cell line, to determine a specificity, if any, of the test compound for affecting DNA or histone methylation, or DNA or histone methyltransferase activity, in the ADK-L cell line relative to the ADK-null cell line.

Paragraph 7. The method of any of paragraphs 1 to 6, further comprising a step of administering the test compound to an animal.

Paragraph 8. The method of paragraph 7, wherein the step of administering is performed if the test compound specifically reduces DNA or histone methylation, or DNA or histone methyltransferase activity, in the ADK-L cell line relative to each of the ADK- null and ADK-S cell lines.

Paragraph 9. The method of any of paragraphs 1 to 8, wherein the step of exposing includes a step of exposing the ADK-L and ADK-S cell lines to a test compound that inhibits DNA or histone methylation.

Paragraph 10. The method of any of paragraphs 1 to 9, wherein the step of exposing includes a step of exposing the ADK-L and ADK-S cell lines to a test compound that binds to ADK.

Paragraph 11. The method of any of paragraphs 1 to 10, wherein each of the ADK- L and ADK-S cell lines expresses the long isoform of an ADK protein or the short isoform of an ADK protein from a respective exogenous construct integrated into the genome of the cell line.

Paragraph 12. The method of any of paragraphs 1 to 11, wherein the step of measuring is performed using an agent that binds specifically to methylated DNA or a methylated form of a histone. Paragraph 13. The method of paragraph 12, wherein the agent is an antibody that binds to 5-methylcytosine.

Paragraph 14. The method of paragraph 13, wherein the step of measuring is performed using DNA or histones from each cell line immobilized on a substrate.

Paragraph 15. The method of any of paragraphs 1 to 14, wherein the step of measuring includes a step of measuring DNA or histone methyltransferase activity in a lysate of a nuclear preparation of each cell line.

Paragraph 16. The method of any of paragraphs 1 to 15, wherein the step of measuring includes a step of performing a sequencing reaction on DNA isolated from each cell line.

Paragraph 17. The method of any of paragraphs 1 to 16, wherein each cell line is a baby hamster kidney cell line.

Paragraph 18. The method of any of paragraphs 1 to 17, wherein the long isoform and the short isoform originate from the same species.

Paragraph 19. A set of cell lines for compound screening, comprising: (A) an ADK-null cell line that does not express any ADK protein; (B) an ADK-L cell line that expresses the long (L), nuclear isoform of a mammalian adenosine kinase (ADK) protein from an exogenous construct; and (C) an ADK-S cell line that expresses the short (S), cytoplasmic isoform of a mammalian ADK protein from an exogenous construct; wherein each copy of an endogenous ADK gene has been inactivated in each cell line.

Paragraph 20. The set of paragraph 19, wherein each of the ADK-L and ADK-S cell lines is a clonal derivative of the ADK-null cell line containing an ADK-L or ADK-S expression construct.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A screening method to identify compounds that alter DNA or histone methylation, the method comprising:
selecting an ADK-null cell line, an ADK-L cell line, and an ADK-S cell line, wherein each copy of an endogenous adenosine kinase (ADK) gene has been inactivated in each of the cell lines, wherein the ADK-null cell line does not express any ADK protein, wherein the ADK-L cell line expresses the long (L), nuclear isoform of a mammalian ADK protein from an exogenous construct, and wherein the ADK-S cell line expresses the short (S), cytoplasmic isoform of a mammalian ADK protein from an exogenous construct;
exposing each of the cell lines to the same test compound;
measuring a level of DNA or histone methylation, or DNA or histone methyltransferase activity, for each of the exposed cell lines; and
comparing the level for each exposed cell line to a corresponding level measured without exposure to the test compound, to determine whether the test compound affects DNA or histone methylation, or DNA or histone methyltransferase activity, in any of the cell lines.

2. The method of claim 1, wherein the step of comparing includes a step of determining whether exposure to the test compound increases DNA or histone methylation, or DNA or histone methyltransferase activity, in the ADK-null cell line.

3. The method of claim 1, wherein the step of comparing includes a step of determining whether exposure to the test compound decreases DNA or histone methylation, or DNA or histone methyltransferase activity, in the ADK-L cell line and in the ADK-S cell line.

4. The method of claim 3, wherein the step of comparing includes a step of determining a specificity, if any, of the test compound for reducing DNA or histone methylation, or DNA or histone methyltransferase activity, in the ADK-L cell line relative to the ADK-S cell line.

5. The method of claim 1, wherein each of the ADK-L and ADK-S cell lines is a clone produced by transfection of the ADK-null cell line with an ADK-L or ADK-S expression construct.

6. The method of claim 1, wherein the step of comparing includes a step of comparing the level for the ADK-L exposed cell line with the level for the ADK-null exposed cell line, to determine a specificity, if any, of the test compound for affecting DNA or histone methylation, or DNA or histone methyltransferase activity, in the ADK-L cell line relative to the ADK-null cell line.

7. The method of claim 1, further comprising a step of administering the test compound to an animal.

8. The method of claim 7, wherein the step of administering is performed if the test compound specifically reduces DNA or histone methylation, or DNA or histone methyltransferase activity, in the ADK-L cell line relative to each of the ADK-null and ADK-S cell lines.

9. The method of claim 1, wherein the step of exposing includes a step of exposing the ADK-L and ADK-S cell lines to a test compound that inhibits DNA methylation.

10. The method of claim 1, wherein the step of exposing includes a step of exposing the ADK-L and ADK-S cell lines to a test compound that binds to ADK.

11. The method of claim 1, wherein each of the ADK-L and ADK-S cell lines expresses the long isoform of an ADK protein or the short isoform of an ADK protein from a respective exogenous construct integrated into the genome of the cell line.

12. The method of claim 1, wherein the step of measuring is performed using an agent that binds specifically to methylated DNA.

13. The method of claim 12, wherein the agent is an antibody that binds to 5-methylcytosine.

14. The method of claim 13, wherein the step of measuring is performed using DNA from each cell line immobilized on a substrate.

15. The method of claim 1, wherein the step of measuring includes a step of measuring DNA methyltransferase activity in a lysate of a nuclear preparation of each cell line.

16. The method of claim 1, wherein the step of measuring includes a step of performing a sequencing reaction on DNA isolated from each cell line.

17. The method of claim 1, wherein each cell line is a baby hamster kidney cell line.

18. The method of claim 1, wherein the step of measuring includes a step of measuring a level of histone methylation or histone methyltransferase activity for each of the exposed cell lines.

19. A set of cell lines for compound screening, comprising:
an ADK-null cell line that does not express any ADK protein;
an ADK-L cell line that expresses the long (L), nuclear isoform of a mammalian adenosine kinase (ADK) protein from an exogenous construct; and
an ADK-S cell line that expresses the short (S), cytoplasmic isoform of a mammalian ADK protein from an exogenous construct;
wherein each copy of an endogenous ADK gene has been inactivated in each cell line.

20. The set of claim 19, wherein each of the ADK-L and ADK-S cell lines is a clonal derivative of the ADK-null cell line containing an ADK-L or ADK-S expression construct.
